(19) United States
(12) Patent Application Publication
(10) Pub. No.: US 2021/0000757 A1
(43) Pub. Date: Jan. 7, 2021

(54) TOPICAL COMPOSITION

(71) Applicant: Futura Medical Developments Limited, Guildford Surrey (GB)

(72) Inventor: Adrian Davis, Guildford Surrey (GB)

(21) Appl. No.: 16/639,435

(22) PCT Filed: Aug. 16, 2018
(86) PCT No.: PCT/GB2018/052323

§ 371 Date: Feb. 14, 2020

(30) Foreign Application Priority Data
Aug. 16, 2017 (GB) 1713160.8

(51) Int. Cl. A61K 31/04; A61K 9/06; A61K 47/32; A61K 47/10; A61P 15/10

(52) U.S. Cl. CPC A61K 31/04; A61K 9/06; A61P 15/10; A61K 47/10; A61K 47/32

(57) ABSTRACT

There is described a composition for topical application to the penis for treatment of erectile dysfunction, the composition comprising glyceryl trinitrate (GTN) as active ingredient dissolved in a blend of volatile and non-volatile solvents of different solvating capacities for the GTN, in which the volatile solvents comprise water and a lower alcohol and the non-volatile solvents comprise a polyhydric alcohol and a glycol having a ratio by weight of from 1.5:1 to 6.0:1, in which the composition has a pH as manufactured in the range 5.1 to 7.0. Also described is the use of the composition and methods involving the composition for treating or ameliorating erectile dysfunction.

### FIELD OF THE INVENTION

This invention relates to topical compositions for the treatment or amelioration of conditions susceptible to vascular smooth muscle relaxation, especially erectile dysfunction.

### BACKGROUND TO THE INVENTION

Erectile dysfunction (ED) is a sexual dysfunction characterised by the inability to develop or maintain an erection of the penis during sexual activity in humans. The pathology of ED is mainly linked to cardiovascular, neurological or psychological factors, or to a combination of these. First-line treatment is with an oral PDE5 inhibitor such as Sildenafil, Tadalafil or Vardenafil, which inhibits the metabolism of the erectile stimulant nitric oxide—cyclic GMP system within the penis. However, side effects of oral PDE5 inhibitors include headache, flushing and dizziness and are due to vasodilatory effects on the capillary smooth muscle in other parts of the body.

Current second-line treatments of ED include regional intracavernosal needle injection or intraurethral suppository or cream application of Alprostadil (synthetic prostaglandin E1) to the penis, again to stimulate the nitric oxide-cyclic GMP system. However, Alprostadil is associated with local irritant effects, such as pain or a burning sensation at the site of application—these effects also being experienced by the sexual partner when the composition is in the form of a cream—and in any event is chemically unstable, such that refrigeration is required for storage purposes.

It is clear, however, that the pharmacokinetics and biopharmaceutics of oral PDE5 inhibitors are not ideal for the treatment of ED. Oral formulations with more rapid absorption, for example, Sildenafil, or newer PDE5 molecules with longer systemic half life, such that future sexual encounters may be planned for, have been developed. Even so, the side effects of oral PDE5 inhibitors remain a problem, in main part driven by their long plasma half life. Nevertheless, the time frame of sexual encounters is usually measured in minutes, for example the duration of the sexual act from penetration to ejaculation is around 8 minutes, on average. In general, the biopharmaceutical requirement for a rapid onset of activity and the sustainment of activity (usually measured in hours, not minutes) results in a design conflict, such that neither requirement is fully met.

Additionally, the current first- and second-line treatments for ED have the disadvantage that, in the context of sexual intercourse between consenting and mutually-participating partners, the element of spontaneity is largely lost, potentially resulting in an unsatisfactory or unfulfilling sexual experience for one or both partners which itself can inhibit or otherwise compromise the success of future sexual occurrences. Overall, therefore, a product which affects the nitric oxide-cyclic GMP system via direct, regional application to the penis has the potential for reduced adverse effects arising from the presence of the compound in the systemic circulation, and the provision of a rapid onset of action. Furthermore, not only would such a product not be required to be applied in advance of sexual activity but also participation in its application during and as part of such activity could be carried out by either, or both, partners. In short, whereas current treatment of erectile dysfunction uses an oral PDE5 inhibitor or an injection or suppository, there is potentially considerable advantage to be gained from the use of a topical composition which does not require refrigeration on storage and can be applied directly to the penis.

Glyceryl trinitrate (GTN) is a nitric oxide donor; a prodrug of nitric oxide, which has been used for example in the treatment of angina and, following topical application, for the treatment of anal fissure. Within the corpus cavernosa of the penis, nitric oxide activates guanylate cyclase, resulting in an increase in cGMP which in turn leads to vasodilation, veno-occlusion and erection. A priori, penile metabolism of GTN is essential to initiate and sustain an erection. Also, Van Ahlen et al. (J. Urol. 1994, 73(3), 316-8) conclude that penile “first-pass” metabolism (of Alprostadil into the inactive 15 keto 13,14 dihydro compound) is an important factor in the lack of significant systemic adverse effects seen with penile application of vasoactive compounds.

Clearance of GTN, and other vasoactive compounds, from the penis is also affected by various erectile processes which impede the venous return of blood to the systemic circulation. Cawello et al. (J. Urol. 1997, 158, 1403-1407), demonstrated that erection results in reduced systemic amounts of the vasoactive compound Alprostadil following its local penile administration. In the non-erect state, penile clearance of vasoactive compounds by venous return is a rapid process which, for GTN, may be at least as important as penile metabolism in the overall penile clearance process. Thus, in the design of a topically applied vasoactive formulation, for example of GTN, required to target delivery to the penis, it is important that onset of erection is rapid, thus to minimise systemic distribution. Of course, rapid onset of erection is also a benefit to users and their partners.

Topical application of a vasoactive compound such as GTN in a semi-solid preparation where the product is applied on the finger of the user or his partner to the glan penis is associated with several benefits. Yang C C and Bradley W E (Br J. Urol. 1998, 82(1), 109-13) concluded that “The distinct pattern of innervation of the glans emphasises the role of the glans (penis) as a sensory structure.” Thus, following stimulation of the glans penis, the dorsal nerve of the penis immediately orchestrates nitric oxide synthesis within the penile cavernosa. It is important, to optimise the combined effect of endogenous and GTN-derived exogenous nitric oxide in order to minimise systemic distribution (and maximise penile targeting), that GTN delivery from the formulation into the penis is also extremely rapid. In the case where loss of volatile solvents is required to form the non-volatile drug delivery engine, shear upon application, application rub-in time and choice of polymeric matrix are all important design considerations.

GTN metabolism within the smooth muscle of the penis is dependent upon the enzyme aldehyde dehydrogenase. Such metabolism-activation is essential for GTN mediated cyclic GMP-mediated vascular relaxation and an erectile response. Berretta et al. (J Biol Chem. 2008, 283(26), 17873-17880) demonstrated that in vitro the peak rate of mitochondrial and cytosolic aldehyde dehydrogenase-mediated formation of nitric oxide from GTN occurs after approximately 100 seconds and then rapidly decreases. In vivo, in man, plasma GTN has a half life of approximately 2-5 minutes suggesting that the rapid decrease of nitric oxide formation seen in vitro may be due to GTN depletion by metabolism. Thus, even where GTN is maintained substantially within the penis, clearance by metabolism is still fairly rapid.

Consideration of these principles leads to the hypothesis that a biphasic delivery of GTN may be required for optimal targeting of GTN to the penis. The hypothesis suggests that, following onset of application of the product, it is firstly essential that very rapid first phase delivery of GTN is needed such that the combined endogenously and exogenously derived nitric oxide work together to maximise onset of erection, thus to minimise systemic absorption. Secondly, once erection is achieved, a second stage of GTN input is required to counter the effects of penile depletion of GTN by metabolism. Upon ejaculation the complex biology of this process works to return the penis to the flaccid state.

The time frame of these processes measured in seconds and single-digit minutes requires exceptional attention to detail in the design of the biopharmaceutics of the product.

WO2006/016139 discloses a topical composition including GTN as the active ingredient for the treatment of erectile dysfunction. In the compositions as disclosed, the GTN is dissolved in a blend of volatile and non-volatile solvents of different solvating capacities for the GTN, the composition being applied in use by hand to the glans and along the shaft of the penis, thereby providing an extended surface area over which the composition is supported and which, in combination with body warmth, will cause the volatile solvent to evaporate. As the volatiles evaporate, the thermodynamic activity of the GTN increases in the residual solvent and, as the GTN passes through the skin and is absorbed in the blood stream, it becomes depleted in the residual composition. The thermodynamic activity of the GTN is maintained against this depletion process by continuing evaporation of the volatile solvent. Typically, volatile solvents are water and a lower alcohol, for example ethanol, and non-volatile solvents comprise a polyhydric alcohol, for example glycerol, together with optionally a minor amount of additional solvent comprising a glycol, for example propylene glycol. The initial objective behind such compositions was to provide a “virtual injection” of the GTN, whereby the effect of the GTN was experienced locally at the region of administration with rapid onset but systemic distribution and resulting systemic effects would be substantially avoided because of the low dose of GTN thus applied.

In practice, it was found that one particular composition described in WO2006/016139, identified as QS13, proved to be an efficient delivery system for GTN. Depending on the dose of GTN, its thermodynamic activity in the residual phase, which controls the permeation through the skin, is up to two times the saturation level, thus providing very good GTN permeation. The QS13 formulation as disclosed contained 10% GTN on lactose, the GTN itself being present at 10% of the lactose-containing composition whereby the concentration in the total formulation was 1%. Whereas this is a relatively low concentration compared with other compositions which had been tried at the time, it was nevertheless considered, despite the self-limiting effect referred to above, that it would be desirable further to reduce the concentration of GTN (that is, the input rate when applied to the penis) in order to minimise possible adverse consequences arising for example from distribution in the systemic circulation.

Composition QS13 itself had included, as excipients, water (35.08%), ethanol (27.01%), glycerol (26.61%), Carbopol® 937P (1.00%) as a homopolymer gelling agent, triethanolamine (0.2%) as base and Propylparaben (0.1%) as an antimicrobial preservative. Unlike QS13 and some of the other compositions disclosed in WO2006/016139, most compositions included, as co-solvent in the residual phase, propylene glycol. The composition identified as QS6, for example, included water (38.25%), ethanol (28.97%), glycerol (19.41%), and propylene glycol (2.77%), together with the other excipients as described for QS13. In assessing drug transport, the experimental systems QS6 and QS13 were compared with Percutol in an experimental model in which diffusion of GTN from the test solvent through a SAMCO Silastic membrane into a buffered phosphate receptor fluid was assessed over a period of one hour. QS6 and QS13 performed similarly in terms of transport of GTN (in excess of 200 ug/cm²/hour) whereas the Percutol exhibited a transport rate of <100 ug/cm²/hr. However, despite rapid onset of action, the effect over elapsed time from onset was not as good as had been anticipated. WO2007/088327 discloses further formulations (Table 5) in which the glycerol concentration was reduced from 30% (QS13 as in WO2006/016139) through 20% (QS19) to 10% (QS20). QS20 demonstrated the effect of a lower concentration of residual phase solvent for GTN in lowering the saturated GTN concentration and, thus, achieving a higher GTN thermodynamic activity in the residual phase as the volatile solvents evaporate. A comparison of the residual activities of QS13 and QS20 (FIGS. 3 and 4) showed that, at a GTN dose of 2.5 mg per 300 mg gel, the residual activity of QS20 was 22.5 whereas for QS13 it was only 7.5. Thus, by reducing the residual phase concentration, represented by glycerol, to 10%, a significant enhancement in residual phase activity could be achieved.

WO2007/088327 also discloses (Table 7) another formulation (QS22) in which the residual phase is represented by glycerol and propylene glycol, the overall solvents comprising water (35.78%), ethanol (33.02%), glycerol (24.0%) and propylene glycol (6.0%), together with Carbopol® 934P (1.0%) and triethanolamine (0.2%). This composition, therefore, has a higher concentration of residual phase compared with QS20 and thus an expectation of reduced residual phase activity.

When compositions such as disclosed in WO2006/016139 and WO2007/088327 are applied to the penis, it is important that the rate of diffusion of the GTN through the components of the residual phase is greater than the rate of skin penetration, whereby the amount of GTN available for skin penetration and permeation is not limited by the diffusion rate. Furthermore, it would also be desirable for application of such compounds to the penis to be confined essentially to the glans rather than generally also to the shaft. The reason for this is that absorption through the glans is more rapid than through the skin of the shaft, thereby resulting in a faster response time and quicker initial tumescence, with faster depletion on the glans itself, thus lowering the potential for direct contact of GTN with the genitalia of the partner, a desirable albeit not essential requirement. In order to localise the composition essentially to the glans, it would be desirable for the composition to have a viscosity such that, especially at body heat and under shear conditions such as would be generated by rubbing with the fingers, the composition becomes more spreadable but nevertheless remains in the form of a gel, rather than becoming so liquid that it develops fluidity. On the other hand, it had been thought that to increase the viscosity by inclusion of a conventional thickening agent at a higher concentration than already present would have an adverse effect on evaporation of volatile solvents and on diffusion of GTN within the residual phase as the volatile solvent evaporates, and on physical appearance due to phase separation of the hydrophilic polymer in an increasingly anhydrous residual phase.

In summary, despite the efficacy of the compositions disclosed in WO2006/016139 and WO2007/088327 in providing a “virtual injection” effect for GTN, it remains desirable to achieve a follow-up or second stage input of GTN to counter the effect of GTN depletion and to be able to localise application of the composition essentially to the glans of the penis by control of the viscosity without compromising either the ability of the volatile solvents to evaporate on application or the diffusion characteristics of the GTN in the residual phase.

### SUMMARY OF THE INVENTION

In one aspect, the present invention provides a composition for topical application to the penis for treatment of erectile dysfunction, the composition comprising glyceryl trinitrate (GTN) as active ingredient dissolved in a blend of volatile and non-volatile solvents of different solvating capacities for the GTN, in which the volatile solvents comprise water and a lower alcohol and the non-volatile solvents comprise a polyhydric alcohol and a glycol, the polyhydric alcohol and the glycol having a ratio by weight of from 1.5:1 to 6.0:1, characterised that in the composition has a pH in the range 5.1-7.0.

Compositions according to the invention are suitable for localised application to the glans of the penis and provide a hybrid virtual injection-slow absorption topical formulation for the treatment of erectile dysfunction. Controlling the pH to a minimum value of 5.1 results in a viscosity which in use and on application to the glans is believed to provide more rapid initial absorption and a faster response time. By “slow absorption” is meant that the GTN is believed in use to continue to be delivered to the penis following the initial first stage virtual injection delivery, to replenish GTN lost by metabolism, thus sustaining the erection and minimising systemic absorption by occlusion of the venous return bloodstream. Preferably the formulation is in gel, cream or serum form although other forms, such as foams or sprays, may be contemplated and are within the scope of the invention with appropriate additive excipients, for example a propellant gas in the case of a foam or spray. More preferably, the formulation is a gel. In formulation types where the composition has the form of a gel and is applied by hand, viscosities in the range of 125,000-600,000 mPas are appropriate to ensure optimum control of application, as governed by the pH being in the range 5.1-7.0. Preferably, the viscosity should have a value in the range 200,000 to 450,000 mPas, more preferably 250,000-350,000 mPas, for example 300,000 mPas when measured by Brookfield Viscometer, Spindle E, 0.3 RPM, at a temperature of 25° C.

It has been found that the pH affects not only viscosity but also stability and appearance. Regarding viscosity, at a pH of below 5.1 and where the composition is a gel, the viscosity is too low and the composition is too fluid to be applied to the glans without being spread or spontaneously dissipating away from the glans. At pH values greater than 5.1, preferably greater than 5.2, viscosity is acceptable although, at a pH above about 5.8, there is an increasing risk, depending on other ingredients present in the composition, that the thickening or gelling agent will form a precipitate, thus rendering the composition visually unacceptable. Surprisingly, however, even at viscosities higher than 300,000 mPas, it has been found that diffusion of GTN within the residual phase is not adversely affected, with the result that higher viscosities can be tolerated without compromising permeation. Regarding appearance, it has been found that the tendency towards flocculation, arising from or exacerbated by the thickening or gelling agent, is mitigated by the pH. In particular, viscosity is unacceptable at a pH significantly below 5.2 whereas, at a pH greater than 5.8, there is a significant risk of flocculation occurring, resulting in a product which may be regarded by consumers as unacceptable. At a pH up to 7.0, the GTN is stable in the composition for up to at least a year, thus avoiding an absolute need for an excess of GTN to be included in compositions for commercial use, although an excess may still be optionally used as a precaution if desired.

Within the broad pH range of 5.1-7.0, a preferred range is 5.2-6 and a more preferred range is 5.25-5.75. At a pH within this range, viscosity, stability and flocculation are all acceptable and there is no perceived effect on diffusion of GTN within the matrix of the gel, resulting in optimal skin permeation.

The above pH values refer to the composition as manufactured, although there may be a tendency for the pH to drift upwards by up to approximately 0.25 of a pH unit within a few weeks of manufacture. Nevertheless, since higher viscosities can be tolerated, any increase in pH after manufacture is also acceptable.

Because of the tendency for upward drift of pH following manufacture, the more preferred pH range for compositions according to the invention should be regarded as 5.25-6.0 and the upper values of the other ranges should be adjusted accordingly.

Compositions according to the invention may also include, depending on their physical form, at least one additional ingredient selected from: agents for enhancing skin feel, for example a silicone oil composition such as Dimethicone 200; a thickening or gelling agent such as a polyacrylate-based composition; a pH control agent such as triethanolamine or an inorganic base; and an antimicrobial preservative such as methylparaben and/or propylparaben.

Concentrations of the additional ingredients are preferably less than 5% by weight, preferably less than 2% by weight, for example 1.0%. However, the pH control agent is added until the pH is within the desired target range of 5.1-7.0, preferably 5.1-6.0 or more preferably 5.25-5.75, all as manufactured.

Regarding the thickening or gelling agent, it has been found desirable to use a polyacrylate-based composition which is readily dispersible in the solvent blend and which facilitates rapid evaporation of the volatile solvents so as to achieve the equilibrium rate for skin permeation preferably within one minute or more preferably within thirty seconds of application to the penis. Suitable thickening or gelling agents comprise high molecular weight interpolymers of a crosslinked unsaturated carboxylic acid polymer, which may be a homopolymer or a copolymer, and a copolymeric steric stabiliser having hydrophilic and hydrophobic moieties. Preferably, the monomer of the unsaturated carboxylic acid polymer comprises acrylic acid or an alkyl ester derivate thereof, and the steric stabiliser preferably comprises either a block copolymer and/or a random copolymer, the block copolymer preferably comprising a polyester such as 12-hydroxystearic acid as the hydrophobic moiety and polyethylene glycol as the hydrophilic moiety. Preferably, the unsaturated carboxylic acid comprises acrylic acid crosslinked with allyl sucrose. Such interpolymers which include a steric stabiliser in the polymerisation process are rapidly wetted and easy to disperse and, throughout the remainder of this specification, including the claims, will be referred to as “easy to disperse interpolymers of the type described”. Commercially-available representative examples include Carbopol® Ultrez 10, 20, 21 and 30. Homopolymers such as Carbopol® 934P and 937P are not “easy to disperse interpolymers of the type described”, since they do not include the steric stabiliser.

The thickening or gelling agent is preferably present in the composition at 0.5% to 2% by weight. In some embodiments, the composition comprises a thickening or gelling agent at 0.5% to 1.5% by weight. In other embodiments, the composition comprises a thickening or gelling agent at 0.7% to 1.5% by weight. In various embodiments, the composition comprises a thickening or gelling agent at 0.8% to 1.2% by weight. In particular embodiments, the composition comprises a thickening or gelling agent at about 1% by weight.

Compositions according to the invention may be considered as a single-phase solution comprising the volatile solvent pair of, for example, ethanol and water and the non-volatile solvent pair of, for example, glycerol and propylene glycol. The use in such systems of a gelling agent comprising an easy to disperse interpolymer of the type described and having the required pH has been found to permit precise application of the composition to the glans penis, whereupon sensory stimulation of the richly innervated dorsal penile nerve sends signals for nitric oxide synthesis within the corpus cavernosa, resulting in tumescence and erection. Following application to the glans penis, for example by the partner, the volatile solvent pair is lost by evaporation and the concentration of the thermodynamic activity of the glyceryl trinitrate is increased to drive passive diffusion into the glans penis and underlying cavernosal tissue. It is believed that the process of solvent evaporation and delivery of exogenous nitric oxide is sufficiently rapid to coordinate the effect thereof with the nitric oxide produced endogenously by stimulation of the glans, resulting in a synergistic effect arising from the pH and the easy to disperse gelling agent Additionally, the use of easy to disperse interpolymers of the type described, especially Carbopol® Ultrez 10, surprisingly results in higher viscosities than previously imagined being advantageously acceptable, since such interpolymers result in the compositions having rheological properties such that, in use and on being applied to the glans of the penis by hand, the rubbing or smearing action results in a temporary reduction in viscosity such that evaporation of volatile solvent is rendered even more rapid, whereby equilibrium is established in the target time of less than one minute, preferably less than 30 seconds. At the same time, permeation is unaffected by the increased viscosity under non-shear conditions.

Preferably, the polyhydric alcohol and the glycol have a ratio by weight of from 2:1 to 6:1. More preferably, the polyhydric alcohol to glycol ratio by weight is from 2.5:1 to 5.5:1. Even more preferably, the polyhydric alcohol to glycol ratio by weight is from 3:1 to 5:1. More preferably still, the polyhydric alcohol to glycol ratio by weight is from 3.5:1 to 4.5:1.

In terms of concentration of ingredients, compositions according to the invention may comprise the following, the ranges being expressed in percentages by weight of the overall composition:
lower alcohol: 30-45%
water: 20-40%
polyhydric alcohol: 22-26%
glycol: 4-12%

The concentration of water is preferably in the range 30-40% by weight. The 20% referred to above is the minimum which is preferred for gelling purposes. Likewise, the lower alcohol concentration is preferably in the range 30-35% by weight but, subject to user tolerance, concentrations up to 45% may be accommodated without compromising the efficacy of the composition. The ratio of ethanol to water in the volatile solvent pair may be adjusted to alter the rate of evaporation, ethanol being more volatile than water, up to approximately 1:1, the limiting concentration of ethanol as an example of the lower alcohol being governed by local intolerance as a skin irritant.

Preferably, the combined amount of the polyhydric alcohol and the glycol is not more than 35% by weight.

In the present specification, the term “lower alcohol” means an aliphatic alcohol having from one to five carbon atoms, for example, ethanol or isopropanol; ethanol is generally preferred.

By “polyhydric alcohol” is meant an aliphatic polyol such as glycerol, although zorbitol, erythrotol, arabitol and xylitol are examples of other water-soluble polyols which may optionally be used together with or instead of glycerol.

By “glycol” is meant a primary or secondary diol or polyol compound, such as propylene glycol (propene-1,2-diol), butyline glycol (butane-1,3-diol), pentylene glycol (pentane-1,5-diol) or hexyline glycol (2-methyl-2,4-pentane diol).

In compositions according to the invention, the glyceryl trinitrate is preferably supplied as a 5% solution in ethanol and, in the formulations defining the invention, the ethanol content is included in the overall lower alcohol concentration.

Preferably, compositions according to the invention have the following concentrations in percentages by weight:
lower alcohol: 30-35%
water: 33-37%
polyhydric alcohol: 22-26%
glycol: 4-8%

The compositions preferably comprise 0.05% to 1% GTN by weight. More preferably, the compositions comprise 0.1% to 0.8% GTN. More preferably still, the compositions comprise 0.2% to 0.6% GTN.

By way of example, one formulation according to the invention has the following ingredients in percentages by weight:
ethanol: 33%
water: 35%
glycerol: 24%
propylene glycol: 6%
Carbopol® Ultrez 10: 1%
glyceryl trinitrate: 0.2%

The above formulation therefore contains 0.2% glyceryl trinitrate. In some embodiments, glyceryl trinitrate as an ingredient is dissolved in ethanol. If this is the case, the ethanol carrier is included in the overall concentration of ethanol.

By way of example, another formulation according to the invention has the following ingredients in percentages by weight:
ethanol: 33%
water: 35%
glycerol: 24%
propylene glycol: 6%
Carbopol® Ultrez 10: 1%
glyceryl trinitrate: 0.4%

In the above formulations, the ratio of glycerol to polyethylene glycol is 4:1. The above formulations also contain a base to bring the pH within the required range of 5.1-7.0.

Compositions according to the invention may be made by mixing the ingredients together and adjusting the pH to within the target range. Conventional process principles may be applied, for example that the Carbopol® Ultrez 10 is dispersed in the water phase, the ethanol and glyceryl trinitrate added and the remaining solvents then added with or before pH adjustment. While an organic base, such as triethanolamine, may be used for the purpose of pH adjustment, it is preferred to use an inorganic base such as potassium hydroxide, sodium hydroxide or liquid ammonia, to avoid a possibility of nitrosamine formation. Such bases, preferably potassium hydroxide, are especially beneficial in solvent-rich systems such as those according to the present invention, since the potassium, for example, has the potential to form a salt with the gelling agent, the salt possibly being insoluble at the concentrations used, leading to phase separation after manufacture. Easy to disperse interpolymers of the type described appear to be less susceptible to phase separation, due possibly to the increased solvent affinity of the block copolymer backbone segments. Even so, it is considered preferable to control pH to within a range of 5.25 to 5.75 such that precision of application is achieved, Carbopol® salt phase separation does not occur and gel viscosity does not compromise rate of loss of volatile solvents.

Formulations of the current invention are particularly advantageous in that the gel is spread over the richly innervated surface of the glans penis until substantial evaporation of the volatile excipients has occurred. Although this stimulation would be expected to increase any erectile placebo response, formulations according to the invention, because of the processes described above, produce a synergistic increase in rate of onset and duration of erection, typically producing erection within 5 minutes of application.

From the above analysis, it is clear that precise control of the input rate of GTN applied topically into the corpus cavernosa is needed to optimise onset time and duration of efficacy. This precise control is achievable with carefully designed topical GTN delivery technology, as in the present invention.

Also provided is a method for treating or ameliorating erectile dysfunction, the method comprising administering a biologically effective amount of the composition described above to the penis of a subject.

In addition, there is provided the composition defined above for use in treating or ameliorating erectile dysfunction. Also provided is the use of the composition defined above in the manufacture of a medicament for treating or ameliorating erectile dysfunction.

### BRIEF DESCRIPTION OF THE DRAWINGS

The invention will now be described in detail by way of example only with reference to the figures in which:

FIG. 1a shows the mean plasma concentrations of GTN following topical application of a virtual injection formulation (this formulation corresponds to the compositions of WO2006/016139 and is referred to as ‘MED 2003’).

FIG. 1b shows the mean plasma concentrations of GTN following topical application of a hybrid virtual injection-slow absorption formulation (this formulation corresponds to the compositions of the invention and is referred to as ‘MED 2005’).

FIG. 2.1 shows the effect of GTN dose and formulation type on apparent t½.

FIG. 2.2 shows the effect of GTN dose and formulation type on Cmax plasma.

FIG. 2.3 shows the effect of GTN dose and formulation type on Tmax plasma.

FIG. 2.4 shows the effect of GTN dose and formulation type on AUC 0-∞.

### EXPERIMENTAL RESULTS

A pharmacokinetic study measuring systemic plasma levels of GTN following topical application of virtual injection (MED 2003, 0.075, 0.1, 0.25 and 0.50 mg of GTN) and hybrid virtual injection-slow absorption gel formulations of GTN (MED 2005 1.2 mg of GTN) was conducted in 16 volunteers. The MED 2005 formulation contained: ethanol: 33%; water: 35%; glycerol: 24%; propylene glycol: 6%; Carbopol® Ultrez 10: 1%; glyceryl trinitrate: 0.2%. pH was adjusted to 5.25 with potassium hydroxide solution.

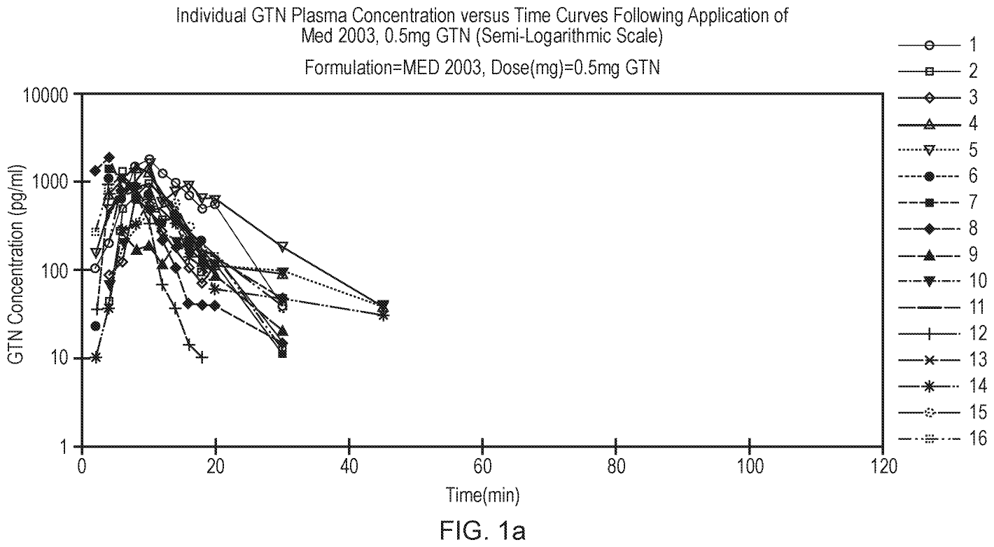

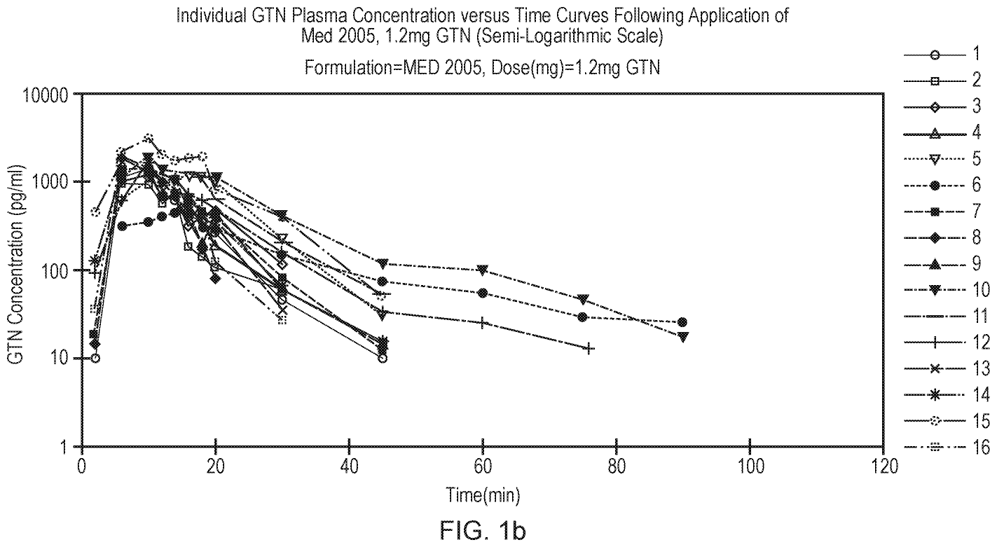

In the laboratory setting of this study, GTN clearance from the penis via the venous return was rapid in the non-erect state. Even so these plasma levels data allow comparison of the pharmacokinetics of GTN from virtual injection and hybrid virtual injection-slow absorption gel formulations. FIGS. 1a and 1b show log individual (n=6) plasma concentrations of GTN with time plots for each volunteer following topical administration of MED 2003, 0.5 mg GTN (virtual injection) and MED 2005, 1.2 mg (hybrid virtual injection-slow absorption) gel formulations, respectively. It is seen that, for some subjects, GTN was present in the plasma for up to 90 minutes following application of the MED2005 gel formulation.

By eye, and by statistical analysis, the apparent plasma t½ (the half life of the apparent elimination phase) is longer for MED 2005.

This finding is consistent with so-called flip-flop pharmacokinetics. Flip-flop pharmacokinetics arise when the absorption rate and elimination rate constants are of similar magnitude, especially when the elimination rate is faster than the absorption rate constant. In this case, the apparent elimination rate is governed by the absorption rate and is thus a characteristic of the formulation. In the Background section, we describe how GTN absorption rate was required to in the same range as the penile clearance rates for GTN.

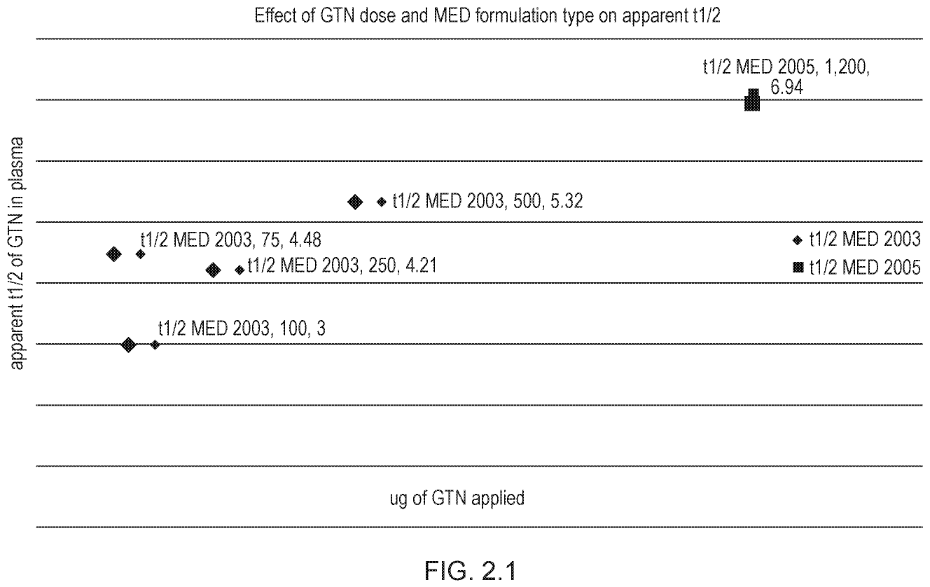

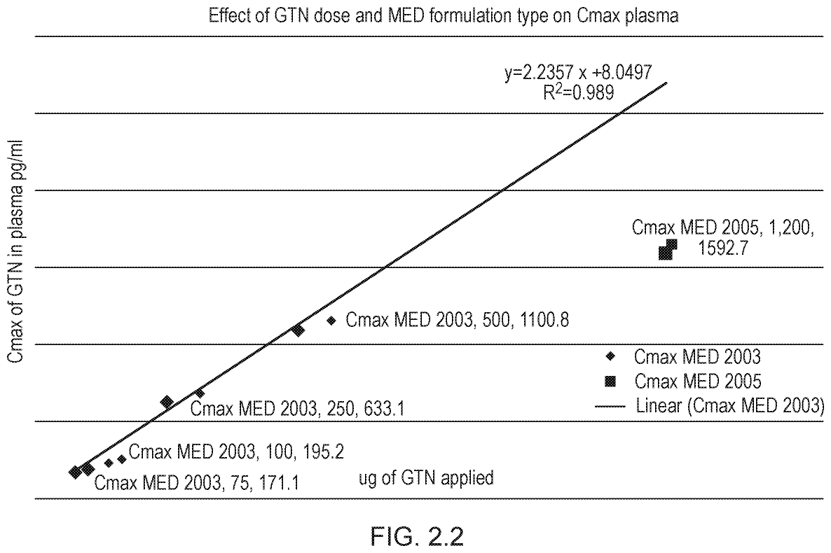

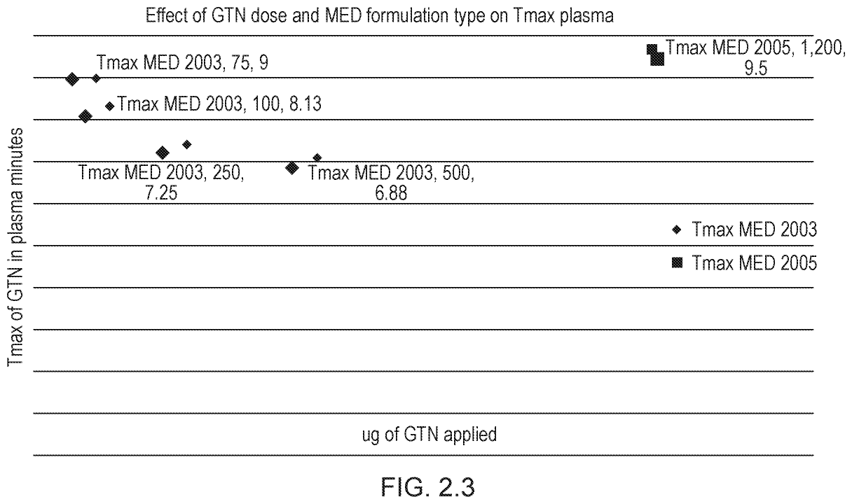

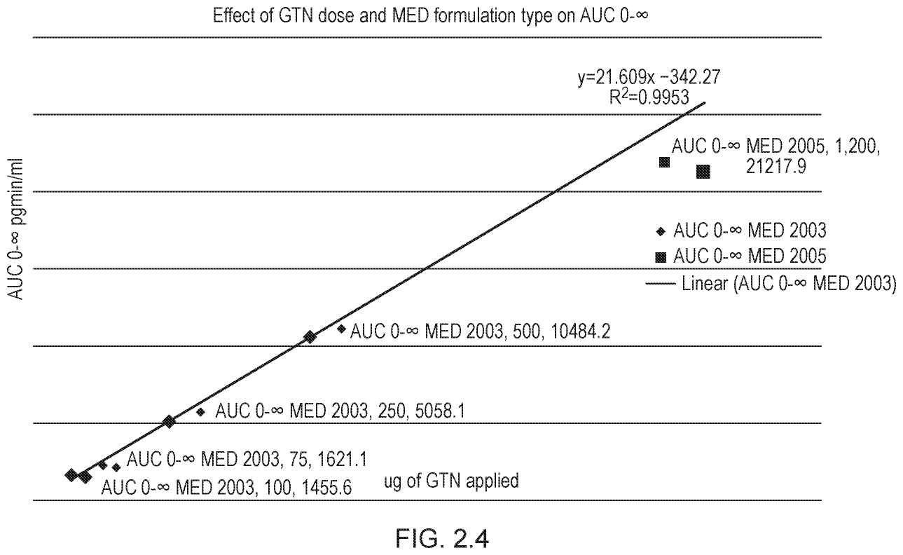

Mean plasma level data with time from several dose of GTN in MED 2003 and a single 1.2 mg dose of GTN in MED 2005 were fitted to a one-compartment pharmacokinetic model and the model-derived pharmacokinetic parameters (t½, apparent systemic half life, minutes; Cmax. maximum plasma concentration, μg/ml; Tmax, time of maximum plasma concentration, minutes and AUC, area under curve 0-∞ pg·min/min) obtained are shown in FIGS. 2.1-2.4. FIG. 2.1 shows that the apparent half life of GTN is increased and FIG. 2.2 that Cmax is decreased in the MED 2005 hybrid virtual injection-slow absorption gel formulation. Both of these pharmacokinetic effects are consistent with slow absorption of GTN from the MED 2005 formulation. FIG. 2.3 shows little effect of MED 2005 formulation on Tmax, which is more typical of a hybrid virtual injection-slow absorption system than one which provides a more steady (slow) absorption. Finally, FIG. 2.4 shows that bioavailability, as measured by AUC 0-∞/dose, is very similar for MED 2003 and 2005 formulations.

These derived pharmacokinetic parameters are consistent with those of a system designed to optimise both onset and duration of activity appropriate to treatment of ED.

### Clinical Studies on Erectile Dysfunction (ED) for MED 2005

Popular culture may regard the successful treatment of ED in terms of the ability to achieve and sustain an erection to enable intercourse to take place. Thus, both onset and duration of activity are important in treatment of ED. In order to assess the degree of severity of ED on a more objective basis, the International Index of Erectile Function (IIEF) has formulated a questionnaire containing questions relating to the evaluation of male sexual function. The erectile function domain of the IIEF questionnaire relates to confidence in achieving and maintaining an erection; whether sexually-stimulated erections are hard enough for penetration; the ability to maintain an erection following penetration; the ability to maintain an erection to completion of intercourse; and how often was the experience of intercourse satisfactory (for the male participant).

In a clinical study, formulations of the invention, containing glyceryl trinitrate, ethanol to water in a ratio of around 1:1 and the Carbopol® Interpolymer Ultrez-10 were remarkably effective, compared to an otherwise identical placebo gel. Particularly, onset of erection occurred with 5 minutes of application. It appears likely that the interpolymer of the type described, for example Carbopol® Ultrez-10, acts in some way to optimise volatile solvent evaporation, either by increasing the rate of total solvent evaporation or preferential loss of ethanol. Shear thinning and reduced viscosity, upon application of the gel, may also contribute to the effect.

In all the above respects, IIEF scores obtained following application of GTN compositions according to the present invention are remarkably high. Also in these clinical studies on compositions of the present invention, subjects reported that time for onset of erection is on a scale of minutes and much shorter than experienced with oral PDE5 inhibitors. We therefore believe that the compositions of the present invention compared with those disclosed in WO2006/016139, provide both initially rapid and yet slowly absorbed delivery of GTN resulting in both rapid onset of erection and appropriate sustainment to the benefit of both sexual partners.

A further pharmacokinetic study has shown that absorption of GTN through the glans of the penis using a composition including Ultrez 10 as the gelling agent follows the usual initial pattern of rapid absorption but in some subjects a “tail” of GTN in the PK profile, lasting up to 4 hours (the duration of the study), is demonstrated. This suggests that the compositions of the invention do provide a hybrid virtual injection-slow absorption effect in prolonging the efficacy time following application. This finding reinforces the inference drawn from FIG. 1b concerning the concentration of GTN in plasma up to 90 minutes from application.

An additional study was carried out to assess the safety and efficacy of compositions according to the invention containing different concentrations of GTN. Doses ranged up to 0.8% (2.4 mg GTN) compared with an orally-administered sub-lingual composition (Nitrostat) at 1.8 mg GTN. Concentrations of GTN and its metabolites were measured in plasma samples using tandem mass spectrometry. In a further study to measure gel absorption, penile swabs were taken for the participants, 5 minutes after application of 0.8% (2.4 mg) of GTN gel in order to measure residual GTN.

It was found that plasma concentration of GTN and its two major metabolites increased proportionally with increasing dosage level. Pharmokinetic modelling suggested that GTN initially enters the systemic circulation by a rapid zero order process with a Tmax of 10-12 minutes. This is followed by a first order absorption with a lag time, the first appearance of GTN being around 4 minutes after application. Comparison of Cmax and the AUC data between compositions according to the invention and the reference product Nitrostat indicated that, at concentrations up to and including 0.6% (1.8 mg) GTN, the bioavailability of systemically absorbed GTN was less than or equivalent to the reference product, indicative of systemic safety. Adverse events experienced by the participants were generally mild and acceptable and, in particular, incidence of headache did not increase markedly at higher doses.

Penile swabs taken 5 minutes after application showed that 73% of the dose was absorbed, suggesting that adverse events as a result of transference to partners would be minimal.

In summary, compositions according to the invention have been shown to have a fast onset of action, low systemic bioavailability and a favourable safety profile for both partners.

1. A composition for topical application to the penis for treatment of erectile dysfunction, the composition comprising glyceryl trinitrate (GTN) as active ingredient dissolved in a blend of volatile and non-volatile solvents of different solvating capacities for the GTN, in which the volatile solvents comprise water and a lower alcohol and the non-volatile solvents comprise a polyhydric alcohol and a glycol having a ratio by weight of from 1.5:1 to 6.0:1, in which the composition has a pH as manufactured in the range 5.1 to 7.0.

2. A composition according to claim 1, having a pH as manufactured in the range 5.25 to 5.75.

3. A composition according to claim 1, having the form of a gel, cream or serum.

4. A composition according to claim 3, wherein the composition has the form of a gel having a viscosity in the range 125,000 to 600,000 mPas.

5. A composition according to claim 4, including at least one additional ingredient selected from agents for enhancing skin feel, a thickening or gelling agent, a pH control agent and an antimicrobial preservative.

6. A composition according to claim 5, wherein the additional ingredient is a thickening or gelling agent which comprises an easy to disperse interpolymer of the type described.

7. A composition according to claim 6, wherein the easy to disperse interpolymer of the type described is a high molecular weight interpolymer of a crosslinked unsaturated carboxylic acid polymer and a copolymeric steric stabiliser having hydrophilic and hydrophobic moieties.

8. A composition according to claim 7, wherein the monomer of the unsaturated carboxylic acid polymer comprises acrylic acid or an alkyl ester derivate thereof.

9. A composition according to claim 7, wherein the unsaturated carboxylic acid comprises acrylic acid crosslinked with allyl sucrose.

10. A composition according to claim 7, wherein the steric stabiliser comprises either a block copolymer and/or a random copolymer, the block copolymer comprising a polyester such as 12-hydroxystearic acid as the hydrophobic moiety and polyethylene glycol as the hydrophilic moiety.

11. A composition according to claim 6, wherein the thickening or gelling agent is present in the composition at 0.5% to 2% by weight.

12. A composition according to claim 1, wherein the lower alcohol is ethanol or isopropanol.

13. A composition according to claim 1, wherein the polyhydric alcohol is selected from glycerol, zorbitol, erythritol, arabitol and xylitol.

14. A composition according to claim 1, wherein the glycol is selected from propylene glycol, butylene glycol, pentylene glycol and hexylene glycol.

15. A composition according to claim 1, wherein the polyhydric alcohol to glycol weight ratio is from 3:1 to 5:1.

16. A composition according to claim 1, comprising the following ingredients, the ranges being expressed in percentages by weight of the overall composition:
lower alcohol: 30-45%
water: 20-40%
polyhydric alcohol: 22-26%
glycol: 4-12%

17. A composition according to claim 1, wherein the water is in the range 30-40% by weight.

18. A composition according to claim 1, wherein the lower alcohol is in the range 30-35% by weight.

19. A composition according to claim 1, wherein the combined amount of the polyhydric alcohol and the glycol is not more than 35% by weight.

20. A composition according to claim 1, wherein the composition comprises a neutralising agent selected from potassium hydroxide, sodium hydroxide or liquid ammonia.

21. A composition according to claim 1, wherein the composition comprises a neutralising agent selected from potassium hydroxide, sodium hydroxide or liquid ammonia, and wherein the composition comprises a thickening or gelling agent which comprises an easy to disperse interpolymer of the type described.

22. A composition according to claim 1, comprising the following ingredients, the ranges being expressed in percentages by weight of the overall composition:
lower alcohol: 30-35%
water: 33-37%
polyhydric alcohol: 22-26%
glycol: 4-8%

23. A composition according to claim 1, wherein the composition is in the form of a gel and comprises the following ingredients, the ranges being expressed in percentages by weight of the overall composition:
lower alcohol: 30-35%
water: 33-37%
polyhydric alcohol: 22-26%
glycol: 4-8%
a thickening or gelling agent: 0.5-1.5%,
wherein the composition has a pH as manufactured in the range 5.25 to 5.75.

24. A composition according to claim 1, wherein the concentration of GTN is in the range 0.05 to 1.0% by weight.

25. A method for treating or ameliorating erectile dysfunction, the method comprising administering a biologically effective amount of the composition according to claim 1 to the penis of a subject.

26. (canceled)
